(19) United States
(12) Patent Application Publication
(10) Pub. No.: US 2015/0297445 A1
(43) Pub. Date: Oct. 22, 2015

(54) NEURONAL INTERFERENCE DEVICES

(71) Applicant: Charles R. Gordon, in his capacity as trustee of the Charles Gordon Trust, Tyler, TX (US)

(72) Inventors: Charles R. GORDON, Tyler, TX (US); Grace E. GORDON, Tyler, TX (US); Reid R. GORDON, Tyler, TX (US); James E. Deaton, Georgetown, TX (US)

(21) Appl. No.: 14/692,045

(22) Filed: Apr. 21, 2015

Related U.S. Application Data
(60) Provisional application No. 61/981,995, filed on Apr. 21, 2014.

(51) Int. Cl. A61H 23/00; A61F 7/08

(52) U.S. Cl. CPC A61H 23/00; A61F 7/08

(57) ABSTRACT

This disclosure includes various vibrating patches that may include: a flexible layer having an adhesive backing and a circuit having a battery, a vibrating device, and an adjustable speed controller having a mechanically adjustable user input device configured to receive user input indicative of a desired frequency of vibration, where the adjustable speed controller is configured to vary the frequency of vibration of the vibrating device at least partly based on the user input. Some of the present vibrating patches include a timing circuit configured to modulate power and/or voltage from the battery to the vibrating device. Others of the present vibrating patches include or are disposed in or within an adjustable headband.

### BACKGROUND

1. Field of Invention

The present invention relates generally to methods and apparatuses for relieving stress, tension, and/or pain, and more specifically, but not by way of limitation, to vibrating patches configured to accomplish the same.

2. Description of Related Art

Examples of vibrating patches are disclosed in U.S. Pat. No. 7,300,409, U.S. patent application Ser. No. 13/201,338, which is published as Pub. No. US 2012/0059294, U.S. patent application Ser. No. 13/746,772, which is published as Pub No. US 2013/0204169, and U.S. patent application Ser. No. 11/566,004, which is published as Pub. No. 2007/0149905.

Vibrating devices have a variety of medical and/or therapeutic uses, ranging from relieving stress, tension, and/or pain, to increasing a user's energy level. Typically, such devices are coupled to a user's skin where they can generate vibrations that are transferred into the user's body.

Circulation is one aspect that has been shown to improve with vibratory stimulation. Increased circulation offers many benefits, for example, reductions in inflammation, faster wound healing, and/or the like. An increase in circulation can also facilitate removal of waste products from muscle tissues, which may reduce muscle soreness after a workout and/or facilitate relief from tense and/or otherwise sore muscles.

Vibrating devices can also be used to provide drug-free pain treatment. Such devices can be placed at or near an afflicted (e.g., painful) area on a user's body, where the vibrations can confuse or overstimulate the user's nervous system which may result in a numbing effect. Furthermore, through proper placement of the devices, pain signals from the afflicted area may be substantially blocked from reaching the user's brain (also known as gate control theory), thus substantially reducing the sensation of pain. Such treated pain can be chronic (e.g., fibromyalgia) or acute (e.g., injections, wound cleaning, and/or the like).

Devices that vibrate can also be used to increase a user's energy level. Some users of vibratory devices have indicated that vibrations can create a total body sense of relaxation. This may be, in part, attributed to other beneficial effects of vibrations, such as increased circulation, relief from stress, tension, pain, and/or the like. Additionally, vibrating devices can mimic the feeling of a massage, which is well known to relieve stress, tension, and/or pain.

Currently available vibrating devices are generally not configured to allow the user to readily adjust the parameters of vibration (e.g., frequency of vibration, amplitude of vibration, and/or the like), and those that are typically require additional and often complex and/or expensive components (e.g., microcontrollers, microprocessors, network components, external controllers, and/or the like) to accomplish this functionality. Such complex and/or expensive components can result in a substantial cost for vibrating devices which may be prohibitively expensive for some and/or vibrating device reuse which can raise serious health concerns (e.g., contamination).

### SUMMARY

Embodiments of the present vibrating patches can be considered “neuronal interference devices” in that they are configured to vibrate to block and/or interrupt signals (e.g., pain signals) from travelling through the nervous system.

Some embodiments of the present vibrating patches are configured, through a mechanically adjustable user input device coupled to an adjustable speed controller, to allow a user to adjust the parameters of vibration (e.g., frequency, amplitude, and/or the like) during use, without the need for microprocessors, processors, network components, external controllers, and/or the like. Some embodiments of the present vibrating patches are configured, through the use of simple (e.g., passive) electronic components, to be disposable (e.g., have a low cost such that it is economically feasible to dispose of the present vibrating patches after use).

Some embodiments of the present vibrating patches comprise a flexible layer having an adhesive backing and a circuit coupled to the layer on a side opposite the adhesive backing, where the circuit comprises a battery, a vibrating device, and an adjustable speed controller having a mechanically adjustable user input device configured to receive user input indicative of a desired frequency of vibration, where the adjustable speed controller is configured to vary the frequency of vibration of the vibrating device at least partly based on the user input. In some embodiments, the circuit comprises a switch configured to selectively activate or deactivate the vibrating device. In some embodiments, the circuit comprises an insulative strip configured to prevent electrical communication through the circuit until removed by a user. In some embodiments, the circuit comprises a heating element. In some embodiments, the battery comprises a button battery.

In some embodiments, the circuit comprises a timing circuit configured to modulate power and/or voltage from the battery to the vibrating device. In some embodiments, the timing circuit comprises an MIC1557 timer. In some embodiments, the timing circuit comprises a 555 timer.

In some embodiments, the vibrating device comprises an electric motor having a shaft and an eccentric weight coupled to the shaft and configured to produce vibrations when the motor is activated. In some embodiments, the vibrating device comprises a piezoelectric vibrator.

In some embodiments, the adjustable speed controller comprises a rheostat. In some embodiments, the adjustable speed controller comprises a potentiometer. In some embodiments, the adjustable speed controller comprises a heat sink configured to dissipate excess power and/or voltage as heat. In some embodiments, the adjustable speed controller comprises a pulse width modulation circuit. In some embodiments, the adjustable speed controller comprises a MAX1749 vibratory motor controller.

In some embodiments, the mechanically adjustable user input device comprises a knob. In some embodiments the flexible layer comprises plastic. Some embodiments of the present vibrating patches are contained within a disposable package.

Some of the present methods comprise coupling a vibrating patch to a user's skin, the vibrating patch comprising a mechanically adjustable user input device configured to adjust a frequency of vibration of the vibrating patch and adjusting the frequency of vibration by adjusting the user input device.

Some of the present vibrating headbands comprise an adjustable headband and a circuit coupled to the headband, where the circuit comprises a battery, a vibrating device, and an adjustable speed controller having a mechanically adjustable user input device configured to receive user input indicative of a desired frequency of vibration, where the adjustable speed controller has a mechanically adjustable user input device configured to receive user input indicative of a desired frequency of vibration and the adjustable speed controller is configured to vary the frequency of vibration of the vibrating device at least partly based on the user input.

The term “coupled” is defined as connected, although not necessarily directly, and not necessarily mechanically; two items that are “coupled” may be unitary with each other. The terms “a” and “an” are defined as one or more unless this disclosure explicitly requires otherwise. The term “substantially” is defined as largely but not necessarily wholly what is specified (and includes what is specified; e.g., substantially 90 degrees includes 90 degrees and substantially parallel includes parallel), as understood by a person of ordinary skill in the art. In any disclosed embodiment, the term “substantially” may be substituted with “within [a percentage] of” what is specified, where the percentage includes 0.1, 1, 5, and 10.

Further, a device or system that is configured in a certain way is configured in at least that way, but it can also be configured in other ways than those specifically described.

The terms “comprise” (and any form of comprise, such as “comprises” and “comprising”), “have” (and any form of have, such as “has” and “having”), “include” (and any form of include, such as “includes” and “including”), and “contain” (and any form of contain, such as “contains” and “containing”) are open-ended linking verbs. As a result, an apparatus that “comprises,” “has,” “includes,” or “contains” one or more elements possesses those one or more elements, but is not limited to possessing only those elements. Likewise, a method that “comprises,” “has,” “includes,” or “contains” one or more steps possesses those one or more steps, but is not limited to possessing only those one or more steps.

Any embodiment of any of the apparatuses, systems, and methods can consist of or consist essentially of—rather than comprise/include/contain/have—any of the described steps, elements, and/or features. Thus, in any of the claims, the term “consisting of” or “consisting essentially of” can be substituted for any of the open-ended linking verbs recited above, in order to change the scope of a given claim from what it would otherwise be using the open-ended linking verb.

The feature or features of one embodiment may be applied to other embodiments, even though not described or illustrated, unless expressly prohibited by this disclosure or the nature of the embodiments.

Some details associated with the embodiments are described above and others are described below.

### BRIEF DESCRIPTION OF THE DRAWINGS

The following drawings illustrate by way of example and not limitation. For the sake of brevity and clarity, every feature of a given structure is not always labeled in every figure in which that structure appears. Identical reference numbers do not necessarily indicate an identical structure. Rather, the same reference number may be used to indicate a similar feature or a feature with similar functionality, as may non-identical reference numbers. The figures are drawn to scale (unless otherwise noted), meaning the sizes of the depicted elements are accurate relative to each other for at least the embodiment depicted in the figures.

FIGS. 1A and 1B depict a top perspective view and a bottom perspective view, respectively, of one embodiment of the present vibrating patches.

FIG. 2 depicts a top perspective view of a headband comprising one embodiment of the present vibrating patches.

FIG. 3 depicts a vibrating device suitable for use in some embodiments of the present vibrating patches.

FIGS. 4A-4E depict circuits suitable for use in some embodiments of the present vibrating patches.

FIG. 5 depicts a top perspective view of a disposable package containing the vibrating patch of FIGS. 1A and 1B.

### DETAILED DESCRIPTION OF ILLUSTRATIVE EMBODIMENTS

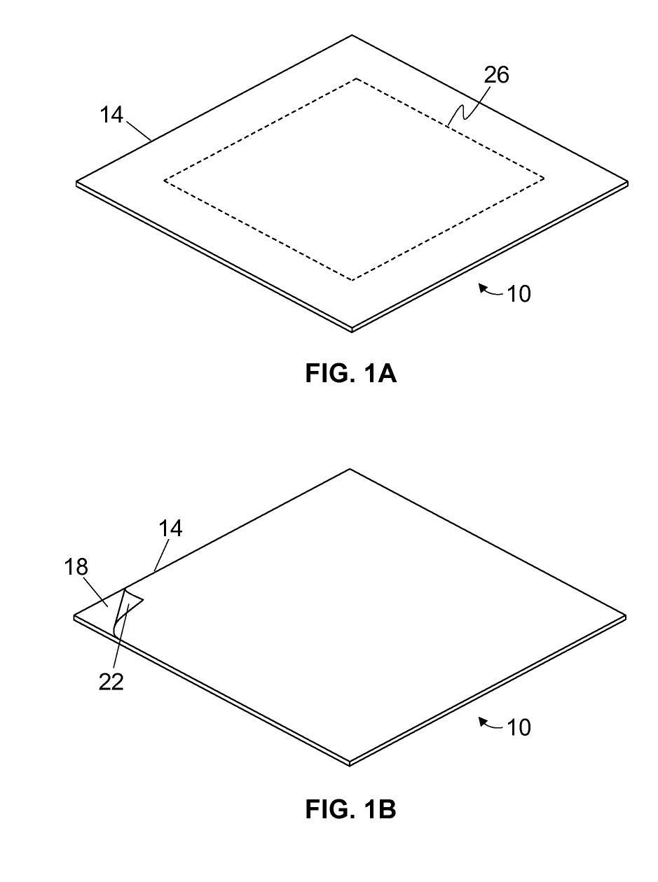

Referring now to the drawings, and more particularly to FIGS. 1A and 1B, shown therein and designated by the reference numeral 10 is a first embodiment of the present vibrating patches. As shown, vibrating patch 10 comprises a flexible layer 14. In this embodiment, the flexible layer comprises plastic; however, in other embodiments, flexible layer 14 can comprise any material which permits the functionality described in this disclosure, including, but not limited to, polymer, rubber, gel or gel-like material, vinyl, paper, fabric, and/or the like. In the embodiment shown, flexible layer 14 (e.g., patch 10) comprises a square shape; however, in other embodiments, flexible layer 14 and/or patch 10 can comprise any shape which permits the functionality described in this disclosure, including, but not limited to, rectangular, triangular, and/or otherwise polygonal, circular, elliptical, and/or otherwise round or rounded. In some embodiments, flexible layer 14 (e.g., patch 10) is configured to be trimmed by a user before use in order to satisfactorily adhere to a desired location on a user's body and/or mitigate undesired interference with the user (for example, when a user desires to place patch 10 on a location on the user's head it may be desirable to trim the patch to prevent interference with eyebrows and/or hair and/or otherwise prevent obstruction of a user's line of vision). In some embodiments, the material properties of layer 14 can be selected to ensure maximum vibrational efficiency and/or to amplify vibrations created by vibrating device 38 (component and placement described in more detail below). For example, the thickness, mass, spring constant, damping coefficient, and/or the like of layer 14 can be selected (e.g., or varied across the layer) to absorb a minimum amount of vibration and/or to amplify the vibrations created by the vibrating device. In this way, the size, power, and/or power requirements of the vibrating device may be minimized, thus reducing the cost of the vibrating patches.

In the embodiment shown, flexible layer 14 comprises an adhesive backing 18. Adhesive backing 18 is configured to releasably secure patch 10 to a desired location on a user's body such that the patch resists inadvertent separation from the user's body when in use (e.g., as a user ambulates and/or otherwise moves), but can be easily removed after use and/or when desired with minimal effort and minimal discomfort to the user (e.g., by minimizing skin and/or hair pulling). Such functionality can be achieved, in part, through selection of the adhesive of adhesive backing 18, which can include, but is not limited to, adhesives currently used in conventional bandages, medical tapes, and/or the like, glues, and/or the like. To ensure that adhesive backing 18 maintains effectiveness (e.g., tackiness) and/or to prevent inadvertent adhesion to a user (e.g., during handling and/or prior to desired placement), patch 10 comprises a removable protective layer 22 disposed on and substantially overlying adhesive backing 18 (e.g., such that patch 10 is not tacky when protective layer 22 is in position, as shown). In the embodiment shown, protective layer 22 is configured to be easily and readily removable (e.g., configured to peel-off, as shown, for example, at a corner) to allow a user to remove protective layer 22, expose adhesive backing 18, and affix patch 10 to a desired part of the body. In the embodiment shown, patch 10 comprises a circuit 26 (e.g., indicated conceptually in FIG. 1A) coupled to layer 14 on a side opposite the adhesive backing (e.g., on an opposite side of the layer from adhesive backing 18).

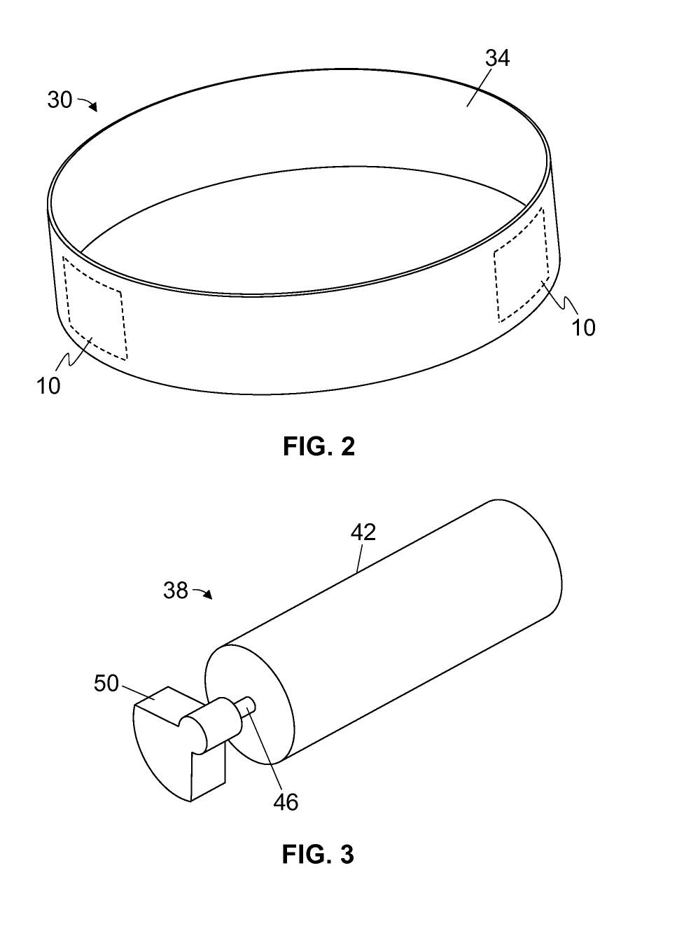

FIG. 2 depicts an embodiment 30 of the present vibrating patches that comprises an adjustable headband 34. In the embodiment shown, headband 34 is configured to be worn on a user's head; however, in other embodiments, the same or a similar adjustable band can be configured to be worn on another part of a user's body (e.g., a leg, arm, torso, and/or the like, for example, by changing the size of the band). In the embodiment shown, headband 34 is adjustable in that the headband comprises an elastic material (e.g., configured to stretch and fit snugly on a user); however, in other embodiments, headband 34 can be adjustable through different and/or additional structure, including, but not limited to, adjustable straps, hook-and-loop fasteners, other fasteners such as buttons, and/or the like. In the embodiment shown, embodiment 30 comprises two (2) vibrating patches 10, that are configured to come in contact with and/or overlie portions of a user's head (e.g., temples) when headband 34 is worn by a user. In this way, vibrations from vibrating patches 10 can be transmitted to a user's head to, for example, facilitate relief from headaches. Embodiment 30 is not intended to be limiting, and any size, material, and/or shape of a band (e.g., headband 34) coupled to any number of vibrating patches is within the scope of the present disclosure. In the embodiment shown, vibrating patches 10 may be coupled to headband 34 through any structure which permits the functionality described in this disclosure, including, but not limited to, adhesives, hook-and-loop fasteners, other fasteners such as snaps or magnets, pockets disposed on and/or within headband 34, coupling members such as clasps and/or the like, and may be internal to headband 34 and/or non-removable (e.g., sewn into headband 34). In embodiments where vibrating patches 10 are coupled to adjustable headband 34 with structure other than adhesive, the vibrating patches may not comprise an adhesive backing 18 or an associated protective layer 22. Other than the forgoing, vibrating patches 10 of embodiment 30 may be the same as or similar to and comprise any of the features and/or functionality of other embodiments of the present vibrating patches described herein.

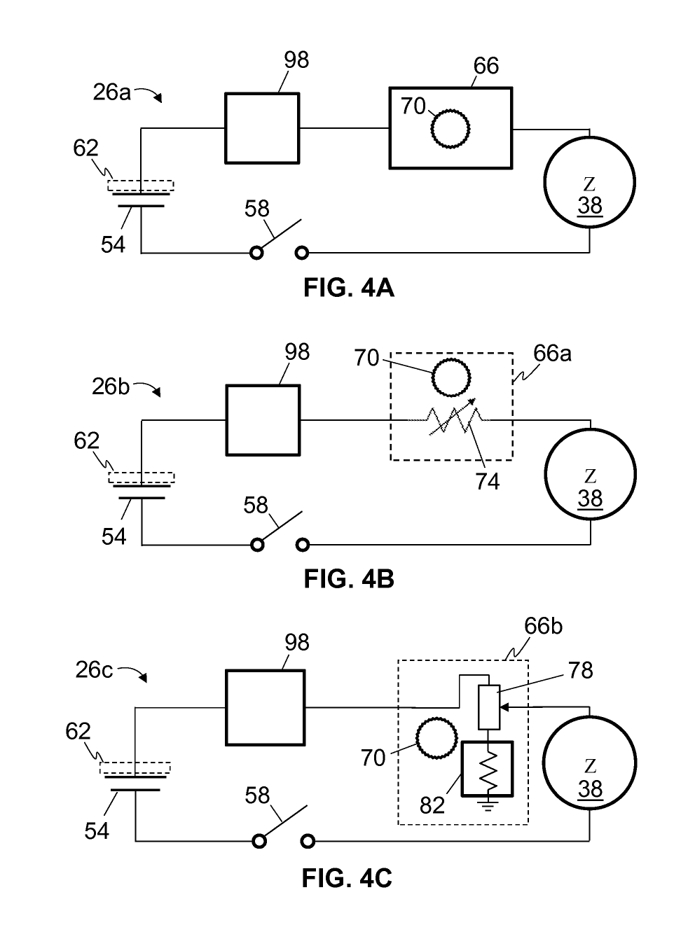

Embodiments of the present vibrating patches can comprise any suitable vibrating device, and one of ordinary skill in the art will understand that the teachings of the present disclosure can apply to any such vibrating device, whether now known or later developed (e.g., by using the same or similar control circuitry, placement, modes of operation, and/or the like as described in the present disclosure). FIG. 3 depicts one example of such a vibrating device 38 which is suitable for use in at least some of the present vibrating patches. In this example, vibrating device 38 comprises an electric motor 42 (e.g., a commercially available “micro” size electric motor). In the embodiment shown, electric motor 42 comprises a shaft 46 and an eccentric weight 50 coupled to the shaft and configured to produce vibrations when the motor is activated. For example, and as shown, eccentric weight 50 is asymmetric about shaft 46 to which it is coupled such that when the shaft spins (e.g., when motor 42 is activated), a rotating unbalance is created due to the uneven distribution of mass about the axis of rotation. Such an unbalance causes vibrations which can then be transmitted to a user wearing the patch via flexible layer 14 and/or patch 10. As discussed above, other embodiments of the present vibrating patches can comprise any vibrating device (e.g., 38) which permits the functionality described in this disclosure (e.g., piezoelectric vibrators, and/or the like).

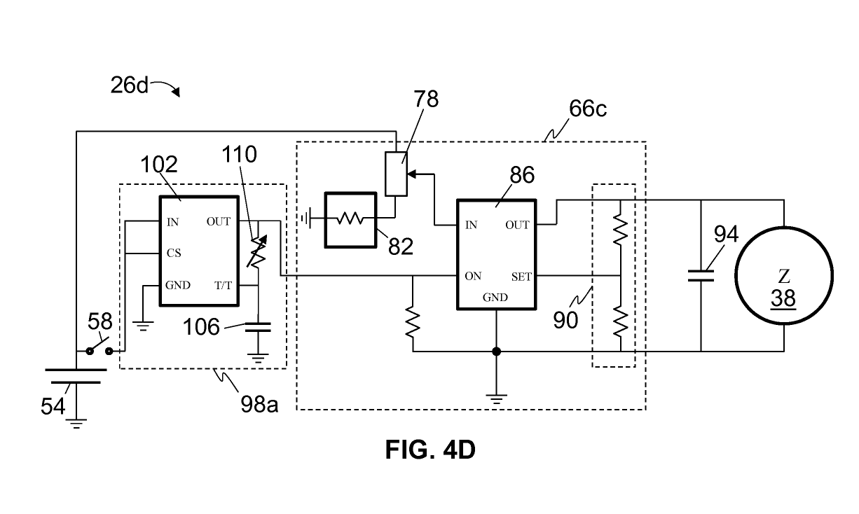

FIG. 4A depicts a circuit 26a that depicts some components present in many embodiments of the present vibrating patches. Embodiments of the present vibrating patches can comprise a variety of circuits and/or electrical components, examples of which are described below with reference to FIGS. 4A-4E. In the embodiment shown, circuit 26a comprises a battery 54. In this embodiment, battery 54 comprises a button battery (e.g., a battery that is substantially flat, such as a conventional hearing aid battery, watch battery, and/or the like). Button batteries are readily available, occupy a relatively small volume (e.g., and can lay flat), and are relatively inexpensive (e.g., to reduce the cost of the present vibrating patches). In the embodiment shown, circuit 26a comprises a vibrating device 38 (an example of which is described above with reference to FIG. 3). In the embodiment shown, circuit 26a comprises a switch 58 configured to selectively activate or deactivate vibrating device 38 (e.g., by selectively allowing or interrupting electrical communication between battery 54 and vibrating device 38). Switch 58 can comprise any suitable structure which permits such functionality, including, but not limited to, conventional switches, sliders, knobs, buttons, and/or the like. Embodiments comprising switch 58 can thus allow the user to selectively activate or deactivate vibrating operation of patch 10 (e.g., to temporarily cease and/or activate vibrations when desired).

In the embodiment shown, circuit 26a comprises an insulative strip 62 configured to prevent electrical communication through circuit 26a (e.g., through placement between battery 54 and one or more associated battery terminals) until strip 62 is removed by a user (e.g., by pulling on the strip and removing it from circuit 26a and/or patch 10). Strip 62 can be configured to mitigate corrosion of terminals associated with battery 54 (e.g., by preventing contact between battery 54 and associated terminals), as well as prevent battery 54 from inadvertently powering motor and thus draining when patch 10 is displayed in a store, stored by a user, and/or otherwise before a user desires to use patch 10. Some embodiments comprising insulative strip 62 may not comprise a switch 58, and strip 62 can perform the function of switch 58 (e.g., on a one-time basis). In such embodiments, the absence of switch 58 can further reduce the cost of the vibrating patches (e.g., by reducing the number of components of the patches). As shown, circuit 26a comprises an adjustable speed controller 66 having a mechanically adjustable user input device 70 (e.g., a knob, slider, multi-positional switch, and/or the like) configured to receive user input indicative of a desired frequency of vibration. Speed controller 66 is configured to vary the frequency of vibration (described in more detail below) of the vibrating device at least partly based on the user input. Through mechanically adjustable user input device 70, a user can adjust the frequency of vibration manually, without need for costly components either external to and/or on or within patch 10 (e.g., without the need for a processor, microprocessor, and/or components for electronic communications to and/or from patch 10) (e.g., all of the components in some embodiments of the present vibrating patches comprise “passive” electronic components). Through such features, a user can directly adjust the frequency of vibration (e.g., during use of patch 10) to achieve a desired level of vibration and therefore stress, tension, and/or pain relief.

In the embodiment shown, circuit 26a comprises a timing circuit 98 configured to modulate (e.g., pulse, vary, and/or otherwise adjust, in an intermittent and/or cyclical fashion) power and/or voltage from battery 54 to vibrating device 38. Through such modulation, battery 54 can be allowed to recover (for example, through the battery recovery effect) in between vibration cycles, thus prolonging battery life and extending the useful life of the present vibrating patches. Embodiments with timer circuit 98 may be configured to allow user adjustment of power and/or voltage modulation time cycles (e.g., through a mechanically adjustable user input device 70, similar to as described for speed controller 66) (e.g., described in more detail below).

FIG. 4B depicts another example of a circuit 26b, which comprises an adjustable speed controller 66a. Adjustable speed controller 66a comprises an adjustable user input device 70 that is coupled (e.g., mechanically and/or electrically) to a rheostat 74. Rheostat 74 can be a variable resistor and can comprise any suitable (e.g., commercially available) rheostat, including, but not limited to, straight or rotary rheostats. By way of example, a user may change the resistance of rheostat 74 by adjusting adjustable user input device 70 (e.g., by rotating a knob or setting the position of a slider or a multi-positional switch, depending on the type of mechanically adjustable user input device and/or rheostat of the particular vibrating patch). As the resistance of rheostat 74 is increased, current flowing to vibrating device 38 is decreased, and therefore, the frequency of vibration is decreased (e.g., in patches comprising an electric motor, the motor will spin more slowly under lower applied currents). Embodiments comprising adjustable speed controller 66a with rheostat 74 can be configured to provide heat to a user's body when the vibrating patch is in use. For example, rheostat 74 can dissipate excess power and/or voltage as heat (e.g., through resistance heating), which can be transmitted to the user through flexible layer 14 and/or patch 10. Thermal energy transfer can be further enhanced through addition of a heat sink (not expressly shown in FIG. 4B) in thermal communication with rheostat 74. As the resistance of rheostat increases (e.g., is increased by a user), vibrating device 38 will vibrate at a lower frequency and more power and/or voltage will be dissipated as heat through the rheostat. In some of these embodiments, this relationship can be used to configure a vibrating patch to generate a specified or specified range of vibrational frequencies and/or a specified or specified range of thermal energies (e.g., heat) (e.g., by configuring allowable resistances of rheostat 74, available power from battery 54, and/or power requirements of vibrating device 38). However, in other embodiments, the present patches can comprise a thermally insulative element configured to prevent heat from rheostat 74 (e.g., or other components of the present patches) from conducting through flexible layer 14 and/or patch 10 and to a user (e.g., the present patches can comprise a thermally insulative material).

FIG. 4C depicts another example of a circuit 26c, which comprises an adjustable speed controller 66b. Adjustable speed controller 66b is substantially similar to adjustable speed controller 66a, with the primary exception that adjustable speed controller 66b comprises a potentiometer 78 in lieu of a rheostat (e.g., 74). Otherwise, the function of adjustable speed controller 66b is substantially similar to as described above. Potentiometer 78 can comprise any potentiometer which permits the functionality described in this disclosure, including, but not limited to, rotary-type or linear-type, and/or the like (e.g., and can be selected based upon the mechanically adjustable user input device 70 of the particular vibrating patch), and can comprise any suitable resistive material, including, but not limited to, carbon, cermet, conductive plastic, wire, and/or the like. Potentiometer 78 can function as a voltage divider, for example, as a user adjusts user input device 70, voltage into adjustable speed controller 66b can be adjustably split by potentiometer 78 between vibrating device 38 and other components (e.g., or dissipated as heat through a heat sink and/or resistor). In some embodiments (e.g., such as the one shown), adjustable speed controller 66b can comprise a heat sink 82 (with internal resistor) configured to receive a portion of the voltage received by the adjustable speed controller (e.g., heat sink 82 or an associated resistor can also be a component of the present patches and/or circuits, for example, as opposed to a component of an adjustable speed controller). Through such features, embodiments of the present patches comprising circuit 26c can be configured to deliver a desired amount of thermal energy (e.g., heat) to a user wearing the present patches (e.g., similar to as described above for circuit 26b). In further such embodiments, mechanically adjustable user input device 70 can be omitted, and potentiometer 78 can be pre-set (e.g., as in a trimpot potentiometer) to provide a specified amount of thermal energy and a specified amount of vibration (e.g., by dividing an available voltage amongst heat sink 82 and vibrating device 38 based on the pre-set setting). In such embodiments, user adjustment of heating and/or vibration may be achieved through a user adjustable input device elsewhere in the circuit and/or coupled to timing circuit 98 (described in more detail below).

FIG. 4D depicts another example of a circuit 26d, which comprises adjustable speed controller 66c. Adjustable user input device 70 can be present in circuit 26d, but is not shown, and various suitable locations for input device 70 are, for example, described below. Adjustable speed controller 66c is similar to adjustable speed controller 66b, with the primary exception that adjustable speed controller 66c comprises a vibratory motor controller 86 (e.g., a MAX1749 vibratory motor controller, available from Maxim Integrated, Inc.). In the embodiment shown, input into vibratory motor controller 86 is controlled by a potentiometer 78 (e.g., which may be coupled to a heat sink 82 and/or include a user adjustable input device 70, as described above); however, in other embodiments, such input control can be achieved through any structure which permits the functionality described in this disclosure (e.g., rheostat(s), other passive component(s), additional controller(s), and/or the like). In this embodiment, output from vibratory motor controller 86 is adjusted with an external resistor-divider 90 (e.g., which may be adjustable via rheostat(s) and/or the like, similar to as described above, and may comprise a user adjustable input device 70). As shown, switch 58 and timing circuit 98a (described in more detail below) are configured to selectively activate or deactivate vibratory motor controller 86. Also shown in circuit 26d is capacitor 94, which is configured to reduce noise resulting from operation of vibrating device 38 and may be found in other circuits of the present vibrating patches (e.g., 26a, 26b, 26c, and/or 26e). In some embodiments of the present vibrating patches, an adjustable speed controller may comprise a pulse width modulation (PWM) circuit to control a vibrating device (e.g., 38); however, in other embodiments, such PWM control may be facilitated and/or achieved by a timing circuit (e.g., 98), as described below.

In the embodiment shown, circuit 26d also comprises timing circuit 98a. In this embodiment, timing circuit 98a comprises an RC timer/oscillator 102 (e.g., a conventional 555 timer or an MIC1557 RC timer/oscillator, available from Micrel, Inc.). In this embodiment, timer/oscillator 102 is in a monostable configuration, with an (e.g., external) capacitor 106 and an (e.g., external) resistor 110 configured to control the output pulse width. As shown, in the depicted embodiment, resistor 110 may comprise a rheostat (e.g., or potentiometer) such that the resistance of the resistor can be varied. Through at least varying the resistance of resistor 110, the output pulse width can be varied, and thus the frequency of modulation of power and/or voltage from battery 54 to vibrating device 38 can be adjusted. Therefore, in embodiments with timing circuit 98a comprising an RC timer/oscillator 102, timing circuit 98a can function as both the timing circuit and the adjustable speed controller (e.g., by allowing for PWM control of vibrating device 38).

FIG. 4E depicts another example of a circuit 26e, which is substantially similar to circuit 26a with the primary exception that circuit 26e comprises a heating element 114. Heating element 114 can be configured to transfer thermal energy to a user by converting electrical energy (e.g., power and/or voltage) from battery 54 to thermal energy via resistive heating. User and/or pre-set control of heating element 114 can be achieved in a similar fashion as to described above (e.g., using a rheostat and/or potentiometer as a voltage divider). Heating element 114 can comprise any resistive material which permits the functionality described in this disclose, including, but not limited to, metal, ceramic, composite, and/or the like. In some such embodiments, circuit 26e and/or patch 10 can comprise a thermally conductive layer (e.g., comprised of carbon fiber, copper, silver, silicon, aluminum, and/or the like) in thermal communication with heating element 114 (e.g., to maximize heat transfer from the heating element to a user's skin).

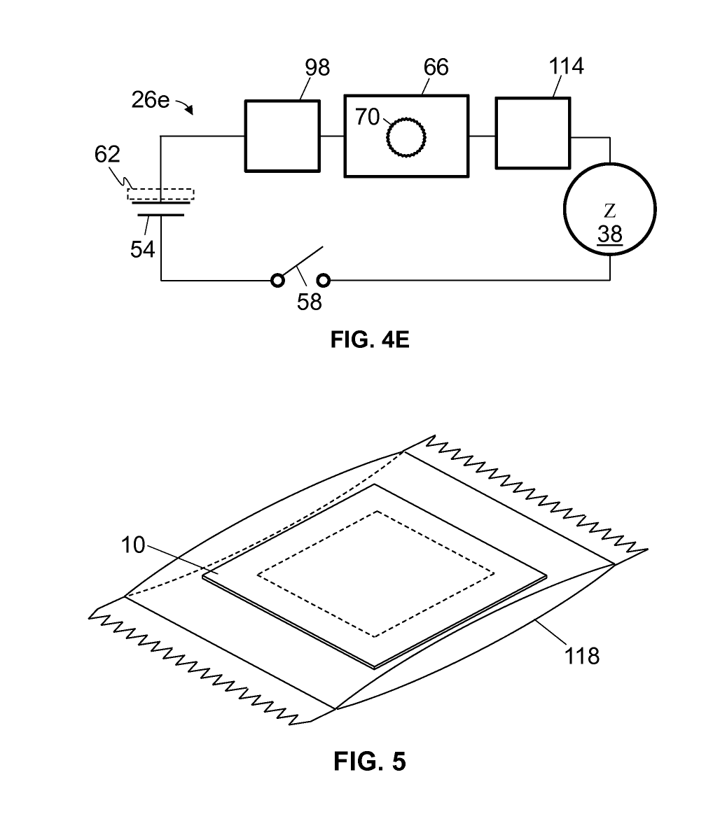

As shown in FIG. 5, some embodiments of the present vibrating patches can be disposed inside of a disposable package 118 (e.g., at the time of purchase and/or before use). In this way, the patches can be sealed to prevent contamination prior to the time of use.

Some of the present methods comprise coupling a vibrating patch (e.g., 10) to a user's body, the vibrating patch comprising a mechanically adjustable user input device (e.g78., 70) configured to adjust a frequency of vibration of the vibrating patch (e.g., as in circuit 26a, 26b, 26c, 26d, and/or 26e), and adjusting the frequency of vibration by adjusting the user input device.

The above specification and examples provide a complete description of the structure and use of illustrative embodiments. Although certain embodiments have been described above with a certain degree of particularity, or with reference to one or more individual embodiments, those skilled in the art could make numerous alterations to the disclosed embodiments without departing from the scope of this invention. As such, the various illustrative embodiments of the methods and systems are not intended to be limited to the particular forms disclosed. Rather, they include all modifications and alternatives falling within the scope of the claims, and embodiments other than the one shown may include some or all of the features of the depicted embodiment. For example, elements may be omitted or combined as a unitary structure, and/or connections may be substituted. Further, where appropriate, aspects of any of the examples described above may be combined with aspects of any of the other examples described to form further examples having comparable or different properties and/or functions, and addressing the same or different problems. Similarly, it will be understood that the benefits and advantages described above may relate to one embodiment or may relate to several embodiments.

The claims are not intended to include, and should not be interpreted to include, means-plus- or step-plus-function limitations, unless such a limitation is explicitly recited in a given claim using the phrase(s) “means for” or “step for,” respectively.

1. A vibrating patch comprising:
a flexible layer having an adhesive backing; and
a circuit coupled to the layer on a side opposite the adhesive backing, the circuit comprising:
a battery;
a vibrating device; and
an adjustable speed controller having a mechanically adjustable user input device configured to receive user input indicative of a desired frequency of vibration;
where the adjustable speed controller is configured to vary a frequency of vibration of the vibrating device at least partly based on the user input.

2. The vibrating patch of claim 1, where the circuit comprises a switch configured to selectively activate or deactivate the vibrating device.

3. The vibrating patch of claim 1, where the circuit comprises an insulative strip configured to prevent electrical communication through the circuit until the insulative strip is removed from the circuit by a user.

4. The vibrating patch of claim 1, where the circuit comprises a timing circuit configured to modulate power and/or voltage from the battery to the vibrating device.

5. The vibrating patch of claim 4, where the timing circuit comprises an MIC1557 timer.

6. The vibrating patch of claim 4, where the timing circuit comprises a 555 timer.

7. The vibrating patch of claim 1, where the battery comprises a button battery.

8. The vibrating patch of claim 1, where the vibrating device comprises:
an electric motor having a shaft; and
an eccentric weight coupled to the shaft and configured to produce vibrations when the electric motor is activated.

9. The vibrating patch of claim 1, where the vibrating device comprises a piezoelectric vibrator.

10. The vibrating patch of claim 1, where the adjustable speed controller comprises a rheostat.

11. The vibrating patch of claim 1, where the adjustable speed controller comprises a potentiometer.

12. The vibrating patch of claim 11, where the adjustable speed controller comprises a heat sink configured to dissipate excess power and/or voltage as heat.

13. The vibrating patch of claim 1, where the adjustable speed controller comprises a pulse width modulation circuit.

14. The vibrating patch of claim 1, where the adjustable speed controller comprises a MAX1749 vibratory motor controller.

15. The vibrating patch of claim 1, where the circuit comprises a heating element.

16. The vibrating patch of claim 1, where the mechanically adjustable user input device comprises a knob.

17. The vibrating patch of claim 1, where the flexible layer comprises plastic.

18. A disposable package containing the vibrating patch of claim 1.

19. A method comprising:
coupling a vibrating patch to a user's skin, the vibrating patch comprising a mechanically adjustable user input device configured to adjust a frequency of vibration of the vibrating patch; and
adjusting the frequency of vibration by adjusting the user input device.

20. A vibrating headband comprising:
an adjustable headband; and
a circuit coupled to the adjustable headband, the circuit comprising:
a battery;
a vibrating device; and
an adjustable speed controller having a mechanically adjustable user input device configured to receive user input indicative of a desired frequency of vibration;
where the adjustable speed controller is configured to vary a frequency of vibration of the vibrating device at least partly based on the user input.
